(19) United States
(12) Patent Application Publication
(10) Pub. No.: US 2016/0279269 A1
(43) Pub. Date: Sep. 29, 2016

(54) METHOD AND REAGENT FOR PREPARING A DIAGNOSTIC COMPOSITION

(71) Applicant: GE HEALTHCARE AS, Olso (NO)

(72) Inventor: Dirk-Jan In't Veld, Oslo (NO)

(21) Appl. No.: 14/777,901

(22) PCT Filed: Mar. 6, 2014
(86) PCT No.: PCT/US2014/021138

§ 371 Date: Sep. 17, 2015

Related U.S. Application Data
(60) Provisional application No. 61/805,556, filed on Mar. 27, 2013.
(60) Provisional application No. 61/839,019, filed on Jun. 25, 2013.

(51) Int. Cl. A61K 49/04

(52) U.S. Cl. CPC A61K 49/0438

(57) ABSTRACT

The present invention provides an aqueous, excipient solution suitable for diluting a diagnostic composition comprising a contrast agent. The excipient solution comprises a sodium ion concentration of about 100-140 mM and a calcium ion concentration of about 0.8-1.2 mM. Alternatively, the molar ratio between sodium ion concentration and calcium ion concentration is between about 80 and 175. Also provided are methods of making and using the excipient solution, as well as a kit comprising the excipient solution.

### FIELD OF THE INVENTION

The present invention is directed to ease individual dosing of X-ray contrast media. More specifically, the present invention is directed to a novel dilution media and a method of using the media for the mixing of concentrated X-ray contrast medium which will result in an isotonic injection. Also provided are a kit and a system for performing the novel method.

### BACKGROUND OF THE INVENTION

All diagnostic imaging is based on the achievement of different signal levels from different structures within the body so that these structures can be seen. Thus in X-ray imaging for example, for a given body structure to be visible in the image, the X-ray attenuation by that structure must differ from that of the surrounding tissues. The difference in signal between the body structure and its surroundings is frequently termed contrast and much effort has been devoted to means of enhancing contrast in diagnostic imaging since the greater the contrast or definition between a body structure or region of interest and its surroundings the higher the conspicuity or quality of the images and the greater their value to the physician performing the diagnosis. Moreover, the greater the contrast the smaller the body structures that may be visualized in the imaging procedures, i.e. increased contrast can lead to increased discernible spatial resolution and conspicuity.

In techniques such as X-ray, one approach for improving the diagnostic quality factor has been to introduce contrast enhancing materials formulated as contrast media into the body region being imaged. Thus for X-ray, early examples of contrast agents were insoluble inorganic barium salts which enhanced X-ray attenuation in the body zones into which they distributed. For the last 50 years the field of X-ray contrast agents has been dominated by soluble iodine containing compounds. Commercial available contrast media (CM) containing iodinated contrast agents are usually classified as ionic monomers such as diatrizoate (Gastrografen™), ionic dimers such as ioxaglate (Hexabrix™), nonionic monomers such as iohexol (Omnipaque™), iopamidol (Isovue™), iomeprol (Iomeron™) and the non-ionic dimer iodixanol (Visipaque™). The clinical safety of iodinated X-ray contrast media has continuously been improved over the recent decades through development of new agents; from ionic monomers (Isopaque™) to non-ionic monomers (e.g. Omnipaque™) and non-ionic dimers (e.g. Visipaque™). However, even the highly refined X-ray contrast media currently on the market exhibit a low degree of undesirable clinical side effects, such as Contrast Induced Nephropathy (CIN), adverse cardiac events, and delayed adverse reactions (DARs). Consequently, there is a clinical need for a new and safer X-ray contrast medium, especially with regards to diagnostic investigations involving patients where there is a high risk of these side effects. Typically one desirable characteristic of X-ray contrast media has been high iodine content, frequently measured in milligrams iodine per milliliter, such as 270-400 mg I/ml. However, to reduce the risk of adverse events, especially in susceptible subjects, to improve patient safety and to reduce costs, there is now a desire to reduce the amount of X-ray contrast media administered to patients undergoing X-ray examinations. At the same time, there is a need for providing contrast media of higher iodine concentrations when needed. Accordingly, there is need for provision of patient friendly X-ray contrast media at various concentrations.

The number of coronary arteriography procedures continues to increase consistent with the expanding capabilities of coronary interventions. In coronary arteriography the blood in the coronary arteries should ideally be completely replaced by a bolus of iodinated radiographic contrast media to maximize the attenuation of radiographs and thereby optimize diagnostic imaging. When contrast media replaces blood, the contrast media molecules cause chemotoxic and osmotic effects in the coronary vessels and also alterations in electrolyte concentrations, viscosity, and oxygen tension. These alterations may influence contractile force and cardiac rhythm and cause ventricular fibrillation (VF). Selective injection of contrast media into the coronary arteries induces regional electrophysiologic and hemodynamic effects. Serious ventricular arrhythmias, as well as cardiodepression, are known complications of coronary arteriography that may be related to the contrast media.

WO91/13636 and WO90/11094, both of Nycomed AS (now GE Healthcare AS), are directed to X-ray contrast media and to their formulations including different salts. There are also numerous studies on the advantages of formulations of X-ray contrast agents with the inclusion of salts, mainly NaCl, in the literature. Of particular relevance is the study by Chai et al. (Acta. Radiol. 2004, 11, 583-593) where it is shown that a formulation of iodixanol containing 19 mM NaCl and 0.3 mM CaCl₂ exhibits a lower frequency of ventricular fibrillation than iodixanol alone.

WO 2009/008734 of GE Healthcare AS discloses a new class of compounds and their use as X-ray contrast agents. The compounds are dimers containing two linked iodinated phenyl groups. The bridge linking the two iodinated phenyl groups is a straight C₃ to C₈ alkylene chain optionally substituted by one to six —OH or OCH₃ groups. A range of compounds are covered by the general formula (I) of the application and many specific compounds are suggested. Compound I, which is one specific dimeric X-ray contrast agent named Ioforminol, falling within the formula I, has been found by the applicant to have favourable properties:

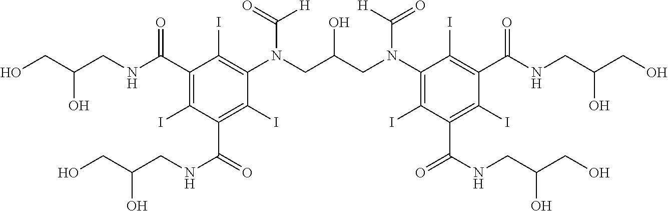

### Compound I: 5-[formyl-[3-[formyl-[3,5-bis(2,3-dihydroxypropylcarbamoyl)-2,4,6-triiodophenyl]amino]-2-hydroxypropyl]amino]-N,N′-bis(2,3-dihydroxypropyl)-2,4,6-triiodobenzene-1,3-dicarboxamide

There is a need for providing patient friendly X-ray contrast media at various concentrations in situ.

### BRIEF SUMMARY OF THE INVENTION

Disclosed herein are novel procedures, systems and excipient solutions for in situ provision of a contrast media at a user defined concentrations. An automated procedure according to embodiments of the current invention provides increased user safety, flexibility and user friendliness.

In one aspect, it is provided an aqueous, excipient solution, which solution comprises a sodium ion and a calcium ion, wherein said excipient solution is suitable for diluting a diagnostic composition comprising a contrast agent.

In another aspect, it is provided a kit, comprising an aqueous, excipient solution according to an aspect of the invention in a first container; a diagnostic composition comprising a contrast agent in a second container; and a user instruction manual.

In a third aspect, it is provided a method for dilution of a diagnostic composition comprising a contrast agent, which method comprises
i) determining a desired dosage amount and dosage concentration for a patient;
ii) calculating a required amount of the diagnostic composition and a required amount of the aqueous, excipient solution according to an aspect of the invention, based on the desired dosage amount and dosage concentration;
iii) delivering the required amount of the diagnostic composition to a mixing chamber; and
iv) delivering the required amount of the excipient solution to the mixing chamber.

In other aspects of the invention, it is provided a method for producing an aqueous, excipient solution according to an aspect of the invention; a method of diagnosis, as well as a method of in vivo imaging detection.

### DETAILED DESCRIPTION OF THE INVENTION

In diagnostic imaging procedures where contrast agents are administered to a patient, it is desirable that the amount of contrast agent used may be adjusted based on the individual patient. Thus, the contrast agent concentration and injection volume best suited for the individual can be achieved. Factors that affect the right concentration and injection volume for any patient may depend on, for example, the type of examination, age, weight or physical health of the patient.

Aspects of the invention provide novel procedures, systems and excipient solutions for in situ generation of a contrast media at a user defined concentrations. Thus, a contrast agent may be manufactured at one higher concentration, and the user (hospital/doctor) may dilute to a desired concentration just prior to use. However, the isotonicity of the solution is maintained throughout the concentration range.

Isotonicity—A solution is isotonic with human blood plasma if no net water migration takes place over the blood cell membranes after mixing the solution with human blood. This means that the measured osmolality of the solution is equal to that of human blood plasma (approx. 290 mOsmol/kg water). This is the goal for any parenteral drug formulation, being more important if injection volumes are relatively large (typically >10 ml) and if injection rate is fast.

Thus, in a first aspect the invention provides an aqueous, excipient solution, which solution comprises a sodium ion and a calcium ion, wherein said excipient solution is suitable for diluting a diagnostic composition comprising a contrast agent. In certain embodiments, the aqueous, excipient solution includes a pharmaceutically acceptable carrier, preferably pure water.

In certain embodiments, the aqueous, excipient solution comprises a sodium ion concentration of about 100-140 mM and a calcium ion concentration of about 0.8-1.2 mM.

In a preferred embodiment, the excipient solution comprises a sodium ion concentration of between about 110-130 mM. In a more preferred embodiment, the excipient solution comprises a sodium ion concentration of between about 115-125 mM. In a most preferred embodiment, the excipient solution comprises a sodium ion concentration of about 119 mM.

In a preferred embodiment, the excipient solution comprises a calcium ion concentration of between about 0.9-1.1 mM. In a more preferred embodiment, the excipient solution comprises a calcium ion concentration of between about 1.00-1.05 mM. In a most preferred embodiment, the excipient solution comprises a calcium concentration of about 1.03 mM.

In one embodiment, the invention provides an aqueous, excipient solution, which solution comprises a sodium ion and a calcium ion, wherein the molar ratio between sodium ion concentration and calcium ion concentration is between about 80 and 175.

In a preferred embodiment, the molar ratio between sodium ion concentration and calcium ion concentration is between about 90 and 130. In a more preferred embodiment, the molar ratio between sodium ion concentration and calcium ion concentration is between about 115-120.

In one embodiment, the sodium ion and calcium ion are from sodium salt and calcium salt comprising a counter ion such as chloride. The selection of a counter ion in the aqueous, excipient solution preferably follows the counter ion used in the X-ray contrast media. In a preferred embodiment, the sodium salt is sodium chloride and the calcium salt is calcium chloride.

In certain embodiments, the excipient solution further comprises an ingredient that protects the contrast agent from degradation. In a preferred embodiment, the ingredient is a pH controlling agent. The pH controlling agent may be a pH buffer. An exemplary pH controlling agent is Tris (tromethamol, THAM). In another preferred embodiment, the ingredient is a chelating agent. An exemplary chelating agent is EDTA (Calcium sodium edetate). In more preferred embodiments, the excipient solution comprises both a pH controlling agent and a chelating agent is EDTA.

An unexpected discovery was made during research in contrast agent formulation that certain contrast agent may be diluted using an aqueous, excipient solution, over a large iodine concentration, while maintaining isotonicity. Thus, with an aqueous, excipient solution of a proper salt combination, a concentrated contrast agent solution may be diluted to any desired iodine concentration for patient administration.

An example of a contrast agent that may suitably be diluted is ioforminol, having the formula:

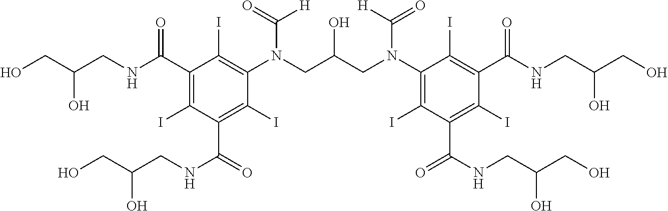 [SMILES: O=CN(CC(O)CN(C=O)c1c(I)c(C(=O)NCC(O)CO)c(I)c(C(=O)NCC(O)CO)c1I)c1c(I)c(C(=O)NCC(O)CO)c(I)c(C(=O)NCC(O)CO)c1I]

Ioforminol may be prepared as outlined in WO 2009/008734. A general procedure is outlined on pages 16-20, and a specific method for preparation is provided in Example 1 of WO 2009/008734. The WO 2009/008734 application, with its description of a process for preparation is hereby incorporated by reference.

Compared to theoretical value where one molecule acts as one hydrated particle in aqueous solution, the compound ioforminol has a lower osmolality. This means that more than one molecule of ioforminol acts as one hydrated particle, indicating a loose form of clustering between single molecules of the compound. Further, there apparently is no dilution effect.

Thus, in certain embodiments, the contrast agent diluted by the aqueous, excipient solution is ioforminol Ioforminol solution of 270 mg I/ml, 320 mg I/ml, or even 350 mg I/ml, may be used as the concentrated contrast agent for dilution, using the aqueous, excipient solution provided according to embodiments of the invention. The excipient solution enables formulation of any concentration of ioforminol between about 70 and 320 mg I/ml by means of in situ dilution from a high concentration ioforminol injection solution available, as long as the high concentration, isotonic ioforminol injection solution has an identical Na/Ca molar ratio.

Similar effect, albeit to a lesser extent, is observed for Iodixanol (Visipaque). Thus, a similar aqueous, excipient solution can be developed for Iodixanol, or any contrast agent that displays a similar effect.

In certain embodiments, the excipient solution contains the same components as a concentrated contrast agent solution, except the contrast agent.

In a particular embodiment, the invention provides an aqueous, excipient solution consisting of Tris (Trometamol) at 10 mM, NaCa-EDTA at 0.27 mM, NaCl at 119 mM and CaCl₂ at 1.03 mM, for diluting a diagnostic composition comprising an ioforminol contrast agent. The excipient solution (i.e., dilution medium) acts as a complement for the ioforminol solution's composition to ensure isotonicity after dilution. Thus, the concentrated ioforminol solution would have the same concentration of Tris and EDTA, but a far lower concentration of NaCl and CaCl₂ in order to be isotonic. The mixing of the concentrated ioforminol solution with the excipient solution in any ratio that generates an ioforminol concentration of 70-320 mg I/ml will yield an isotonic solution as well.

In a second aspect, the invention provides a method for producing an aqueous, excipient solution according to the first aspect of the invention. The method comprises first predicting the molar amount of sodium ion and calcium ion suitable for the excipient using a chemometrical model which describes the linear correlation between contrast agent concentration, salt concentration and osmolality (Y):

Y(osmolality, mOsmol/kg)=0.675×Ioforminol concentration (mg I/mL)+2.78×Na concentration (mM)−47.6;

then making the aqueous, excipient solution according to the predicted molar amount of sodium ion and calcium ion.

In a third aspect, the invention provides a system for the in situ dilution of a contrast agent prior to use. It is provided software, equipment and an aqueous, excipient solution for diluting a concentrated contrast agent solution to any concentration and volume required by any procedure and patient.

Equipment for homogeneous blending of two liquids is well known in the market. For the purpose of preparing a contrast agent solution, it is important that the blending process is performed under sterile condition and the solutions are made of pharmaceutical grade components.

The system for the in situ dilution of a contrast agent also requires a software and algorithm to steer the mixing of the excipient solution and the concentrated contrast agent solution. The software also ensures mixing homogeneity and sterility. Software and algorithms suitable for these applications are well-known.

In another aspect, the invention provides a kit, comprising an aqueous, excipient solution according to certain embodiments of the invention in a first container; a diagnostic composition comprising a concentrated contrast agent solution in a second container; and a user instruction manual.

In certain embodiments, the contrast agent is ioforminol. In a preferred embodiment, the contrast agent has a concentration of between about 70-320 mg l/ml.

In certain embodiments, the aqueous, excipient solution contains the same components as the diagnostic composition, except the contrast agent.

In certain embodiments, the kit further comprises an instrument for homogeneous blending of the aqueous, excipient solution and the diagnostic composition under sterile conditions.

In certain embodiments, the kit further comprises a software that controls the mixing regime resulting in any desired combination of contrast agent concentration and volume.

In another aspect, the invention provides a method for dilution of a diagnostic composition comprising a contrast agent, which method comprises
i) determining a desired dosage amount and dosage concentration for a patient;
ii) calculating a required amount of the diagnostic composition and a required amount of the aqueous, excipient solution, based on the desired dosage amount and dosage concentration;
iii) delivering the required amount of the diagnostic composition to a mixing chamber; and
iv) delivering the required amount of the excipient solution to the mixing chamber.

In certain embodiments, the method for dilution of a diagnostic composition further comprises mixing the diagnostic composition and the excipient solution in the mixing chamber.

In certain embodiments, the desired dosage amount and dosage concentration are determined based in part on the age, weight and physical health of the patient.

The diagnostic composition of the invention is preferably for use as an X-ray contrast media in X-ray diagnoses or X-ray imaging. The composition may be administered as a bolus injection or by infusion. Further, the composition may be administered by intravascular, intravenous or intra-arterial administration. Alternatively, the composition may also be administered orally.

In yet another aspect, the invention provides a method of diagnosis comprising administering a diagnostic composition prepared according to an aspect of the invention to a human or animal body, examining the body with a diagnostic device and compiling data from the examination.

In yet another aspect, the invention provides a method of in vivo imaging detection comprising the following steps;
i) administering a detectable quantity of the diagnostic composition prepared according to an aspect of the invention;
ii) allowing the administered composition to distribute;
iii) detecting signals emitted by the contract agent of the distributed composition,
iv) generating an image representative of the location and/or amount of said signal.

The method of imaging is a method of X-ray imaging and in a preferred embodiment of this aspect, the method of detection is a method of coronary arteriography, and more preferably the diagnostic composition is administered as a bolus injection to the coronary arteries.

### Examples

The following examples are intended only to illustrate methods and embodiments in accordance with the invention, and as such should not be construed as imposing limitations upon the claims.

### Example 1

Ioforminol 320 mg I/ml injection contains 640 mg ioforminol/litre. With a molar weight of 1522,13 Dalton, this would be 420.3 mM. This concentrated solution contains 699 g water per litre. Density is 1.357 kg/litre, thus the weight of one litre Ioforminol 320 mg I/ml injection is 1.357 kg. Water is therefore only about half of the weight. Theoretical estimation of osmolality would be 601.5 mOsmol/kg water given 420.3 mM ioforminol, no dissociation under dissolution and 699 g water as solvent.

Knowing that blood plasma osmolality is approx. 290 mOsmol/kg water, such a solution would be hypertonic by a factor 2 even without other components/solutes. Yet measured osmolality of the complete formulation of ioforminol 320 mg I/ml was around 290 mOsmol/kg water. When one subtracts the contribution to osmolality of the other components in the formulation (Tris, EDTA, NaCl and CaCl₂), the net contribution of ioforminol was around 129 mOsmol/kg, not around 600 mOsmol/kg. This is approximately 22% of the expected theoretical osmolality if every ioforminol molecule would act as one hydrated particle.

This can be attributed to the very high concentration of ioforminol in the 320 mg I/ml solution. Hence one would expect the percentage contribution to osmolality from ioforminol to increase rapidly upon dilution of such a solution from 320 to 70 mg I/ml. However, the percentage only increases to approximately 36% when the concentrated ioforminol composition was diluted more than fourfold. The result of this phenomenon is the linear correlation between ioforminol concentration, salt concentration and osmolality in the interval 70-330 mg I/ml Ioforminol.

Even after autoclaving, a sterilising procedure where aqueous solutions are subjected to 121° C. for approximately 15 minutes, this percentage contribution does not change. Equilibration at high temperatures therewith indicates that the measured osmolality is real and no slow equilibrium causes the unexpected low contribution of ioforminol on osmolality upon dilution.

The effect of this is a linear correlation between ioforminol concentration and NaCl concentration to obtain isotonicity in the range 70-330 mgI/ml.

While the particular embodiment of the present invention has been shown and described, it will be obvious to those skilled in the art that changes and modifications may be made without departing from the teachings of the invention. The matter set forth in the foregoing description and accompanying drawings is offered by way of illustration only and not as a limitation. The actual scope of the invention is intended to be defined in the following claims when viewed in their proper perspective based on the prior art.

1. An aqueous, excipient solution comprising a sodium ion concentration of about 100-140 mM and a calcium ion concentration of about 0.8-1.2 mM, wherein said excipient solution is suitable for diluting a diagnostic composition comprising a contrast agent.

2. The excipient solution according to claim 1, wherein the sodium ion concentration is between about 110-130 mM.

3. The excipient solution according to claim 1, wherein the sodium ion concentration is between about 115-125 mM.

4. The excipient solution according to claim 1, wherein the sodium ion concentration is about 119 mM.

5. The excipient solution according to claim 1, wherein the calcium ion concentration is between about 0.9-1.1 mM.

6. The excipient solution according to claim 1, wherein the calcium ion concentration is between about 1.00-1.05 mM.

7. The excipient solution according to claim 1, wherein the calcium concentration is about 1.03 mM.

8. The excipient solution according to claim 1, wherein the molar ratio between sodium ion concentration and calcium ion concentration is between about 90 and 130.

9. An aqueous, excipient solution comprising a sodium ion and a calcium ion
wherein the molar ratio between sodium ion concentration and calcium ion concentration is between about 80 and 175, and wherein said excipient solution is suitable for diluting a diagnostic composition comprising a contrast agent.

10. The aqueous, excipient solution according to claim 9, wherein the molar ratio between sodium ion concentration and calcium ion concentration is between about 90-130.

11. The aqueous, excipient solution according to claim 9, wherein the molar ratio between sodium ion concentration and calcium ion concentration is between about 115-120.

12. The excipient solution according to claim 1 wherein the sodium ion and calcium ion are from sodium salt and calcium salt comprising counter ions is chloride.

13. The excipient solution according to claim 1 wherein the sodium salt is sodium chloride and the calcium salt is calcium chloride.

14. The excipient solution according to claim 1 further comprising a pH controlling agent.

15. The excipient solution according to claim 14 further comprising a chelating agent.

16. The excipient solution according to claim 14 wherein the pH controlling agent is TRIS and the chelating agent is EDTA.

17. The excipient solution according to claim 1, wherein the solution contains the same components as the diagnostic composition, except the contrast agent.

18. An aqueous, excipient solution consisting of Tris (Trometamol) at 10 mM, NaCa-EDTA at 0.27 mM, NaCl at 119 mM and CaCl₂ at 1.03 mM, for diluting a diagnostic composition comprising a contrast agent.

19. The excipient solution according to claim 1, wherein the contrast agent is ioforminol.

20. A kit, comprising
an aqueous, excipient solution according to claim 1 in a first container;
a diagnostic composition comprising a concentrated contrast agent solution in a second container; and
a user instruction manual.

21. The kit according to claim 20, wherein the contrast agent is ioforminol.

22. The kit according to claim 20, wherein the contrast agent has a concentration of between about 70-320 mg l/ml.

23. The kit according to claim 20, wherein the aqueous, excipient solution contains the same components as the diagnostic composition, except the contrast agent.

24. The kit according to claim 20, further comprising an instrument for homogeneous blending of the aqueous, excipient solution and the diagnostic composition under sterile conditions.

25. The kit according to claim 20, further comprising a software that controls the mixing regime resulting in any desired combination of contrast agent concentration and volume.

26. A method for dilution of a diagnostic composition comprising a contrast agent, which method comprises
i) determining a desired dosage amount and dosage concentration for a patient;
ii) calculating a required amount of the diagnostic composition and a required amount of the aqueous, excipient solution of claim 1, based on the desired dosage amount and dosage concentration;
iii) delivering the required amount of the diagnostic composition to a mixing chamber; and
iv) delivering the required amount of the excipient solution to the mixing chamber.

27. The method of claim 26, further comprising mixing the diagnostic composition and the excipient solution in the mixing chamber

28. The method of claim 26, wherein the desired dosage amount and dosage concentration are determined based in part on the age, weight and health of the patient.

29. A method of diagnosis comprising administering a diagnostic composition prepared according to claim 26 to a human or animal body, examining the body with a diagnostic device and compiling data from the examination.

30. A method of in vivo imaging detection comprising the following steps;
i) administering a detectable quantity of the diagnostic composition prepared according to claim 26;
ii) allowing the administered composition to distribute;
iii) detecting signals emitted by the contract agent of the distributed composition,
iv) generating an image representative of the location and/or amount of said signal.

31. A method as claimed in claim 30 wherein the method is a method of coronary arteriography.

32. A method for producing an aqueous, excipient solution of claim 1, comprising
i) predicting the molar amount of sodium ion and calcium ion suitable for the excipient using a chemiometrical model, based on the salt composition of the diagnostic formulation;
ii) making the aqueous, excipient solution according to the predicted molar amount of sodium ion and calcium ion.
